(19) United States
(12) Patent Application Publication
(10) Pub. No.: US 2011/0183366 A1
(43) Pub. Date: Jul. 28, 2011

(54) Methods for achieving a protective ACE2 expression level to treat kidney disease and hypertension

(76) Inventors: Daniel Batlle, Evanston, IL (US); Minghao Ye, Chicago, IL (US); Jan Wysocki, Chicago, IL (US)

(21) Appl. No.: 12/932,435

(22) Filed: Feb. 25, 2011

Related U.S. Application Data
(62) Division of application No. 11/542,348, filed on Oct. 2, 2006.
Application No. 11/542,348 is a continuation-in-part of application No. PCT/US2005/011190, filed on Apr. 1, 2005.
(60) Provisional application No. 60/558,718, filed on Apr. 1, 2004.

(51) Int. Cl. C12Q 1/37

(52) U.S. Cl. 435/24

(57) ABSTRACT

The present invention provides a method for enhancing expression of angiotensin converting enzyme ACE2 in the vasculature of a mammal, particularly in the renal vasculature and podocytes. The method comprises administering to a mammal in need of such enhancement (e.g., a mammal suffering from, or at risk of developing renal damage or hypertension), an amount of an angiotensin II antagonist sufficient to promote a protective level of ACE2 expression in the vasculature of the mammal. Preferably, the angiotensin II antagonist is administered in an angiotensin II blocking amount, more preferably in an amount sufficient to achieve and maintain a desired level of ACE2 expression in the vasculature of the mammal. The methods of the invention are useful for ameliorating kidney damage from diseases, such as diabetes, as well as hypertension.

### CROSS-REFERENCE TO RELATED APPLICATIONS

This application is a division of U.S. Ser. No. 11/542,348, filed on Oct. 2, 2006, which, in turn, is a continuation-in-part of International Application Serial No. PCT/US2005/011190, filed on Apr. 1, 2005, and which claims the benefit of U.S. Provisional Application Ser. No. 60/558,718, filed on Apr. 1, 2004, all of which are incorporated herein by reference.

### FIELD OF THE INVENTION

The invention relates to methods for ameliorating renal damage in mammals. More particularly, the invention relates to methods for maintaining a level of angiotensin converting enzyme 2 expression in a mammalian kidney sufficient to protect the kidney from renal damage associated with diseases such as diabetes.

### BACKGROUND OF THE INVENTION

Alterations within the renin-angiotensin system (RAS) are considered to be pivotal for the development of diabetic complications, in particular diabetic renal disease and hypertension. The angiotensin-converting enzyme (ACE), a key element of RAS, is primarily a membrane-bound protein residing on the surface of epithelial and endothelial cells. Through its two catalytic domains, ACE cleaves the inactive precursor angiotensin I (ANG I) to angiotensin II

(ANG II), which induces vasoconstriction, aldosterone release, and acts as growth modulator. Most tissue beds, including the kidney, express a local RAS that acts independently of the circulating system. There is also a growing body of evidence, that implicates the more recently characterized peptides angiotensin (1-7) and angiotensin (3-8) as additional bioactive components of the RAS.

ACE is a monomeric, membrane-bound, zinc- and chloride-dependent peptidyl dipeptidase that catalyzes the conversion of the decapeptide ANG Ito the octapeptide ANG II by removing a carboxy-terminal dipeptide. ACE2 is the only known and enzymatically active homologue of ACE in the human genome. ACE2 is a carboxypeptidase that preferentially removes carboxy-terminal hydrophobic or basic amino acids. Angiotensin I and II, as well as numerous other biologically active peptides, are substrates for ACE2, but bradykinin is not. While ACE is ubiquitously distributed, ACE2 was initially found to be restricted to the heart, kidney, and testis. More recently it also has been found in the colon, small intestine, and ovary, for example.

ACE2 contains only a single enzymatic site that is capable of catalyzing angiotensin Ito angiotensin (1-9). It also degrades ANG II to the vasodilator ANG (1-7), and this may counterbalance the ANG II-forming activity of ACE. In contrast to ACE, ACE2 activity is not inhibited by ACE inhibitors.

Previous studies using the streptozotocin (STZ) model of diabetes revealed decreased renal expression of ACE. A recent study using this rat diabetic model showed a reduction in ACE2 as well. These previous studies involved diabetic rats with advanced renal lesions. The db/db mouse is a genetic model of type 2 diabetes caused by an inactive mutation of the leptin receptor gene that results in a shorter intracellular domain of the receptor and a failure to transduce signals. As a result of this mutation, hyperglycemia develops in association with insulin resistance and obesity around 4-7 weeks after birth. The db/db mouse eventually develops some, but not all, features of human diabetic nephropathy such as renal hypertrophy, glomerular enlargement, and albuminuria. Renal histology evaluation, moreover, shows lesions exhibiting expansion of extracellular matrix as well as augmented laminin chain content. These lesions, however, are not present early on, but rather develop in older animals (by about 20 weeks of age).

There is an ongoing need and desire for improved treatment and prevention of renal failure particularly in diabetics. The present invention fulfills that goal.

### SUMMARY OF THE INVENTION

The present invention provides a method for enhancing expression of angiotensin converting enzyme ACE2 in the vasculature of a mammal, e.g., in the kidneys. The method comprises administering to a mammal in need of such enhancement (e.g., a mammal suffering from, or at risk of developing vascular damage), an amount of an angiotensin II antagonist sufficient to promote a protective level of ACE2 expression in the vasculature of the mammal. Preferably, the angiotensin II antagonist is administered in an angiotensin II blocking amount, more preferably in an amount sufficient to maintain a protective level of ACE2 expression in the vasculature of the mammal.

In a preferred embodiment, the invention provides for a renoprotective level of ACE2 expression in the kidneys, particularly in the renal vasculature and podocytes.

In another preferred embodiment, the invention provides a method for enhancing the expression ratio of ACE2 to ACE in mammalian renal vasculature and podocytes. This method comprises administering to the mammal an angiotensin II blocking amount of an angiotensin II antagonist. Preferably, the ratio of ACE2 expression to ACE expression is increased within the renal vasculature and podocytes.

Preferred angiotensin II antagonists useful in the methods of the present invention include telmisartan, physiologically acceptable salts thereof, and the like.

The methods of the present invention are useful for ameliorating renal damage in mammals, particularly mammals suffering from type 2 diabetes.

In another aspect, the present invention provides a method for ameliorating proteinuria in a mammal suffering from proteinuria. The method comprises administering to the mammal a proteinuria ameliorating amount of an angiotensin II antagonist.

In yet another aspect, the present invention provides an assay method for concurrently determining ACE and ACE2 activity in a tissue sample. The method comprises contacting a first aliquot of a clarified, diluted tissue homogenate with a fluorescent substrate of ACE and ACE2, such as 7-methoxycoumarin-Tyr-Val-Ala-Pro-(2,4-dinitrophenyl)Lys (SEQ ID NO: 7), in a suitable physiological buffer, in the presence of a specific ACE inhibitor, such as captopril, in an amount sufficient to suppress fluorescence that would have resulted from substrate cleavage products formed by reaction of the substrate with ACE present in the aliquot. Subsequently, the fluorescence resulting from cleavage of the substrate by ACE2 in the sample is measured (preferably within about 4 hours) to determine the ACE2 activity in the tissue sample. A second aliquot of the clarified, diluted tissue homogenate is contacted with the ACE/ACE2 substrate in the presence of a specific ACE2 inhibitor (e.g., MLN-4760) in an amount sufficient to suppress fluorescence that would have resulted from ACE2 present in the aliquot. Subsequently, the fluorescence from substrate degradation products formed by reaction of the substrate by ACE in the aliquot is measured (preferably within about 4 hours) to determine the ACE activity in the tissue sample. The ACE and ACE2 activities are each preferably determined by comparison to fluorescence measurements obtained from substrate degradation products formed by reaction of the substrate with suitable standard samples of ACE and ACE2 (e.g., using a calibration curve or the like) under the same assay conditions.

### BRIEF DESCRIPTION OF THE DRAWINGS

FIG. 1 illustrates kidney and heart ACE mRNA levels in db/m and db/db mice. Top panels (Panel A) show kidney cortices from 6 db/m mice (lanes 1-6) and 5 db/db mice (lanes 7-11). (Panel B) shows heart samples from db/m mice (lanes 1-5) and db/db mice (lanes 6-10). Bottom panels show graphs of ACE and GAPDH levels in the mice, indicating that the ACE:GAPDH ratio in kidney cortices (Panel A) were markedly reduced in db/db mice (dark bars) compared to db/m mice (light bars), whereas the ACE:GAPDH ratio in hearts from db/db and db/m mice (Panel B) were similar. Data are provided as mean±standard error (SE).

FIG. 2 illustrates kidney and heart ACE2 mRNA levels in db/m and db/db mice. RNA was isolated from kidney (Panel A) or heart (Panel B) and subjected to RT-PCR for ACE2 and GAPDH. Top panels show kidney cortices from 5 db/m mice (lanes 1-5) and 5 db/db mice (lanes 6-10) (Panel A), and heart tissue from 5 db/mice (lanes 1-5) and 5 db/db mice (lanes 6-10) (Panel B). Bottom panels show the ACE2:GAPDH ratios were not significantly different between db/db mice (dark bars) and db/m mice (light bars) for either kidney (Panel A) or heart (Panel B).

FIG. 3 illustrates ACE activity in kidney cortex and heart in db/m and db/db mice. Panel A shows that ACE activity was markedly lower in kidney cortices from db/db mice (dark bars, n=8) compared to db/m mice (light bars, n=9). Panel B shows that ACE activity in the heart was not significantly different between db/db mice (dark bars, n=8) and db/m mice (light bars, n=9).

FIG. 4 shows kidney ACE and ACE2 protein levels in db/m and db/db mice. Top Panel shows Western blots of membrane protein preparations from renal cortices of 5 db/m mice (lanes 1-5) and 5 db/db mice (lanes 6-10). After probing with ACE (Panel A) or ACE2 (Panel B) antibodies, the blots were reprobed for β-actin. Bottom panel demonstrates, by densitometry, that the ACE: β-actin ratio (Panel A) was markedly reduced in db/db mice (dark bars) compared to db/m mice (light bars). In contrast to ACE, ACE2:β-actin ratio (Panel B) was markedly increased in db/db mice.

FIG. 5 illustrates heart ACE and ACE2 protein levels in db/m and db/db mice. Top panel shows heart ACE protein (Panel A) and ACE2 protein (Panel B) as determined by Western blotting. Bottom panel shows, by densitometry, that ACE and ACE2 protein expression did not differ between db/m (1-5) and db/db mice (6-10).

FIG. 6 illustrates the immunohistochemistry of renal tissue in db/m and db/db mice. Kidney sections were stained for ACE (A, B) and ACE2 (C, D). Renal cortical tubules from the db/db mice (B) exhibit much weaker ACE staining compared to tubules of control mice (A). In contrast, in renal tubules from the db/db mice (D), there was increased ACE2 staining in the apical border as compared to tubules from control mice (C). Micrographs were taken at 200× magnification.

FIG. 7 shows immunohistochemical staining of ACE (A, B) and ACE2 (C, D) in kidney sections from control (A, C) and diabetic mice (B, D). In diabetic mice, there is high intensity of ACE staining in the glomeruli (B, wide arrow) accompanied by weak staining in the proximal tubules (B, narrow arrow) compared to control (A). The reverse was observed with ACE2 staining—in diabetic mice there is little ACE2 staining in the glomeruli (D, wide arrow), accompanied by strong staining in the proximal tubule (D, narrow arrow) compared to the control (C). There is also ACE2 staining in glomerular parietal epithelium from diabetic mice (D, double arrows).

FIG. 8 shows a graph of percentage of glomeruli with strong staining for ACE and ACE2 in control mice (white bars) and diabetic mice (black bars).

FIG. 9 shows immunofluorescence staining of ACE (A) and ACE2 (B) in kidney proximal tubules from db/m mice. ACE staining (gray areas of panel A) is seen only at the brush borders of the proximal tubules. ACE2 staining (gray areas in panel B) was seen mainly at the brush borders and also weakly in the cytoplasm (B, wide arrow). A merged image (C) of panels A and B shows colocalization of ACE and ACE2 (bright areas at arrow in pane C) at the apical level of proximal tubules.

FIG. 10 shows triple immunofluorescence staining of ACE (A, light gray areas), ACE2 (D, gray areas), and AQP2 (B, E, gray areas) to localize ACE and ACE2 in principal cells of collecting tubules from db/m mice. ACE weakly colocalized with AQP2 (C, arrows), while ACE2 exhibited strong colocalization with AQP2 (F, arrows).

FIG. 11 shows immunofluorescence staining of ACE (A, gray areas) and ACE2 (B, gray areas) in glomeruli from db/m mice kidney. Panel C shows a merged image of panels A and B indicating no colocalization of ACE and ACE2 in the glomeruli.

FIG. 12 shows triple immunofluorescence staining of ACE (A, light gray areas), ACE2 (D, gray areas), and PECAM-1 (B, E, dark gray areas) to localize ACE and ACE2 in the endothelial cells of the glomerular tuft from db/m mice. ACE strongly colocalized with PECAM-1 (C, light gray areas), while ACE2 did not (F).

FIG. 13 shows triple immunofluorescence staining of ACE (A, light gray areas), ACE2 (D, gray areas), and nephrin (B, E, dark gray areas) to localize ACE and ACE2 in the slit diaphragm from db/m mice. ACE2 strongly colocalized with nephrin (F, light gray areas), while ACE did not (C).

FIG. 14 shows triple immunofluorescence staining of ACE (A, light gray areas), ACE2 (D, gray areas), and podocin (B, E, dark gray areas) to localize ACE and ACE2 in the basal pole of podocytes from db/m mice. ACE2

FIG. 15 shows triple immunofluorescence staining of ACE (A, light gray areas), ACE2 (D, gray areas), and podocin (B, E, dark gray areas) to localize ACE and ACE2 in the basal pole of podocytes from db/m mice. ACE2 weakly colocalized with podocin (F, arrow), while ACE did not (C).

FIG. 16 shows triple immunofluorescence staining of ACE (A, G light gray areas), ACE2 (B, E, gray areas), and PECAM-1 (D, H, dark gray areas) to localize ACE and ACE2 in renal vessels from db/m mice. ACE and ACE2 did not colocalize in the renal vessel (C) in contrast to the proximal tubules (C, bright areas, arrow). ACE colocalized with PECAM-1 in the endothelial layer (I, light gray areas, arrow), while ACE2 did not (F).

FIG. 17 shows triple immunofluorescence staining of ACE (A, light gray areas), ACE2 (B, gray areas), and von Willebrand factor, VWF (C, D, dark gray areas) in renal vessels of db/m mice. ACE is present in tunica intima and is not colocalized with VWF in tunica media (F, arrows).

### DETAILED DESCRIPTION OF PREFERRED EMBODIMENTS

Antagonists of angiotensin II are a class of antihypertisive agents that block access of angiotensin II to its type 1 receptor in preference to the type 2 receptor. The angiotensin II type 1 receptor is important in the regulation of blood pressure and is widely distributed in the kidneys, including in the renal vessels, afferent and efferent artierioles, tubular cells and juxtaglomerular cells. Selectively blocking the type 1 receptor results in changes in renal hydrodynamics (e.g., vasodilation resulting in decreasing renal vascular resistance) and increased sodium excretion. Angiotensin II antagonists inhibit the renin-angiotensin-aldosterone (RAA) system, which is important in blood pressure regulation. In contrast, ACE inhibitors act earlier in the RAA system, actually preventing the formation of angiotensin II, altogether. Thus, ACE inhibitors indirectly inhibit effects at both the angiotensin II type 1 receptor and the type 2 receptor. Because of the selectivity for type 1 receptor inhibition, angiotensin II antagonists do not enhance prostaglandin synthesis or inhibit bradykinin metabolism, both of which effects are observed in patients treated with ACE inhibitors.

Several angiotensin II antagonists have been approved for use in the treatment of hypertension or are under investigation as antihypertensive agents, including, without limitation, losartan, valsartan, irbesartan, candesartan, telmisartan, zolarsartan, tasosartan and eprosartan. Prodrugs of angiotensin II antagonists have also been investigated. Such prodrugs are enzymatically cleaved, in vivo, to form the active drug. An example of an angiotensin II antagonist prodrug is candesartan cilexetil, which reportedly is completely converted to candesartan in the gastrointestinal tract. The degree of affinity for the type 1 receptor relative to the type 2 receptor varies greatly among angiotensin II antagonists. Valsartan reportedly has about 20,000 times greater affinity for the type 1 receptor relative to the type 2 receptor, whereas telmisartan reportedly has about 3,000 times greater affinity for the type 1 receptor versus the type 2 receptor.

As used herein, the term angiotensin II antagonists encompasses free base compounds, physiologically acceptable salts thereof and prodrugs that are cleaved in vivo to form the active angiotensin II antagonist compound.

In another aspect, the present invention provides a method for ameliorating proteinuria in a mammal suffering from proteinuria (e.g., albuminuria). The method comprises administering to the mammal a proteinuria ameliorating amount of an angiotensin II antagonist, such as those described herein.

The methods of the present invention utilize angiotensin II antagonists to maintain a renoprotective level of ACE2 expression in the kidneys and to ameliorate proteinuria, which can result from an ACE2 deficiency. In particular, the methods of the present invention maintain a renoprotective level of ACE2 in the renal vasculature and podocytes and reduce proteinuria by administering an angiotensin II antagonist to a mammal in need of renal protection, such as a mammal suffering from type 2 diabetes. Preferably, the mammal is a human.

In yet another aspect, the present invention provides an assay method for concurrently determining ACE and ACE2 activity in a tissue sample. The method comprises contacting a first aliquot of a clarified, diluted tissue homogenate with a fluorescent substrate of both ACE and ACE2 in a physiologically acceptable buffer in the presence of a specific ACE inhibitor for a time period sufficient to form an amount of a fluorescent substrate degradation product in the aliquot that is proportional to the ACE2 activity in the tissue sample. A second aliquot of the clarified, diluted tissue homogenate is also contacted with the fluorescent substrate of both ACE and ACE2 in the buffer, but in the presence of a specific ACE2 inhibitor, for a time period sufficient to form an amount of a fluorescent substrate degradation product in the aliquot that is proportional to the ACE activity in the tissue sample. Subsequently, the fluorescence from each aliquot is measured. The amount of fluorescence observed in the first aliquot is directly proportional to the ACE2 activity in the tissue sample, while the amount of fluorescence in the second aliquot is directly proportional to the ACE activity in the tissue sample. The ACE and ACE2 activities are each preferably determined by comparison to fluorescence measurements obtained from suitable standard samples of ACE and ACE2 (e.g., using a calibration curve or the like) under the assay conditions.

The clarified, diluted tissue homogenate can be prepared by homogenizing a tissue sample in a physiologically acceptable buffer, clarifying the resulting homogenate by removing solid materials (e.g., by centrifugation), and diluting the resulting clarified homogenate with an additional amount of a buffer.

Suitable buffers include physiologically tolerable buffers having a pH of about 6 to about 7.5. The buffer is selected to be compatible with ACE and ACE2. Such buffers are well known in the art and include, for example, a HEPES-based buffer (pH 7.4), a 4-morpholinoethanesulfonic acid-based buffer (pH 6.5), and the like.

A preferred fluorescent substrate of both ACE and ACE2 for use in the present assay method is 7-methoxycoumarin-Tyr-Val-Ala-Pro-(2,4-dinitrophenyl)Lys (SEQ ID NO: 7; also referred to herein as 7-Mca-YVADAPK(Dnp)).

A preferred specific inhibitor of ACE2 for use in the assay method of the present invention is MLN-4760, (S,S)-2-{1-carboxy-2-[3-(3,5-dichlorobenzyl)-3H-imidazol4-yl]-ethylamino}-4-methylpentanoic acid, available from Millennium Pharmaceuticals. A preferred ACE inhibitor is captopril.

The fluorescence can be determined in any suitable fluorometer. In a preferred embodiment, aliquots of clarified, diluted tissue homogenates from a plurality of tissues are placed in the wells of a multiwell plate so that ACE and ACE2 activity from a plurality of tissue samples can be determined contemporaneously and automatically using a fluorometric plate reading device. Suitable such devices are well known in the art.

Preferably the fluorescence from each aliquot is measured not more than about 4 hours after the contact of the homogenate with the substrate, so as to obtain optimal fluorescence in the sample.

The following examples and discussion are provided to illustrate various aspects of the invention and are not meant to be limiting.

### Example 1

### Quantification of ACE and ACE2 in the Kidney

Animal Model and Biochemical Measurements. Diabetic mice (db/db) were used as a model of type 2 diabetes and their lean litermates (db/m) served as non-diabetic controls (Jackson lab). The db/db mouse is one of the best characterized and most extensively studied rodent models of type 2 diabetes. Heterozygous db/m litermates are lean and are spared from the induction of type 2 diabetes and its secondary complications. As such, the db/m mouse is an ideal genetic control for the db/db mouse. We used only young (8 weeks of age) female db/db mice to study an early phase of diabetes (3 to 4 weeks of onset) without renal complications. The Institutional Animal Care and Use Committee of Northwestern University approved all procedures.

RNA Isolation and RT-PCR. Total RNA was extracted from mice kidney cortices, hearts and lungs with TRIZOL Reagent (Invitrogen). cDNA's were synthesized from 1.0 μg of total RNA by using Access RT-PCR system (Promega) as per manufacturer's instructions and GenAmp PCR System 9700 (Applied Biosystems). The primers used for ACE were 5′TAACTCGAGTGCCGAGGTC-3′ (sense) (SEQ ID NO: 1) and 5′-CCAGCAGGTGGCAGTCTT-3′ (antisense) (SEQ ID NO: 2), corresponding to nucleotide positions 200-218 and 522-539, respectively (ACC #BC040404). ACE2 primers were: 5′-CTTCAGCACTCTCAGCAGACA-3′ (sense) (SEQ ID NO: 3) and 5′-CAACTTCCTCCTCACATAGGC-3′ (antisense) (SEQ ID NO: 4), corresponding to nucleotide positions 489-509 and 899-919, respectively (ACC #BC026801). Glyceraldehyde-3-phosphate dehydrogenase (GAPDH) was used as an internal control for each PCR reaction. GAPDH primers were: 5′-CCAGTATGACTCCACTCACGGCA-3′ (sense) (SEQ ID NO: 5) and 5′-ATACTTGGCAGGTTTCTCCAGGCG-3′ (ACC #NM008084) (SEQ ID NO: 6). The bands corresponding to PCR products were measured by densitometry.

Membrane Protein Preparation and Western Blot Analysis. Membrane proteins from kidney cortices and hearts were isolated and subjected to Western blot analysis as previously described. For detection of ACE, nitrocellulose membranes were incubated with mouse monoclonal antibody (Chemicon). ACE2 protein in kidney tissue was measured using an affinity purified rabbit anti-ACE2 antibody. For heart tissue, we used a commercial ACE2 antibody (Santa Cruz). Signals on Western blots were quantified by densitometry and corrected for β-actin.

ACE Activity Assay. Isolated kidney cortices, hearts and lungs were homogenized in an assay buffer consisting of: (in mmol/L) 50 HEPES, pH 7.4, 150 NaCl, 0.5% Triton X-100, 0.025 ZnCl₂, 1.0 PMSF and then clarified by centrifugation at 10,000×g for about 15 minutes. ACE activity against a synthetic substrate (p-hydroxybenzoyl-glycyl-L-hisidyl-L-leucine) was determined using a colorimetric method (Fujirebio Inc.). For the assay, tissue samples were standardized to 1 μg protein/μl. Optical density was read at 505 nm with a spectrophotometer. Results were calculated as mIU per mg of protein. All data are reported as mean±SE.

Immunohistochemistry. Kidneys were cut and fixed in 10% buffered formalin and embedded in paraffin. Four-μm sections were deparaffinized in xylene and rehydrated through graded alcohols. Antigen retrieval was performed with a pressure cooker at 120° C. in target retrieval solution (DAKO). Endogenous peroxidase activity was blocked with 3% hydrogen peroxide. Slides were then incubated with the same antibodies as described above (anti-ACE or anti-ACE2), and with secondary antibody conjugated with peroxidase-labeled polymer (DAKO). After incubation with DAB+chromogen, slides were counterstained with Hematoxylin. Sections were dehydrated, covered with Permount (Fisher Scientific) and a coverslip, and viewed with a Zeiss microscope.

Animal Characteristics. The basic animal characteristics are shown in Table 1. As expected, db/db mice were much heavier than their lean db/m litermates and had markedly elevated serum glucose levels. Serum cholesterol and triglycerides were also markedly increased. Kidney weight was increased in db/db mice while the kidney to body weight ratio was reduced in db/db mice likely reflecting their larger size.

TABLE 1
Animal Characteristics

| | Control (n = 11) | Diabetic (n = 10) | |
|---|---|---|---|
| Characteristic | db/m mice | db/db mice | p values |
| Body weight (g) | 20.1 ± 0.3 | 34.3 ± 0.4 | <0.0005 |
| Kidney weight (mg) | 0.228 ± 0.010 | 0.260 ± 0.006 | <0.005 |
| Kidney/body weight | 1.1 ± 0.01 | 0.8 ± 0.01 | <0.0005 |
| ratio (%) | | | |
| Serum glucose (mg/dl) | 168 ± 9 | 460 ± 44 | <0.0005 |
| Serum cholesterol | 75 ± 3 | 126 ± 11 | <0.0005 |
| (mg/dl) | | | |
| Serum triglycerides | 179 ± 24 | 265 ± 38 | <0.05 |
| (mg/dl) | | | |

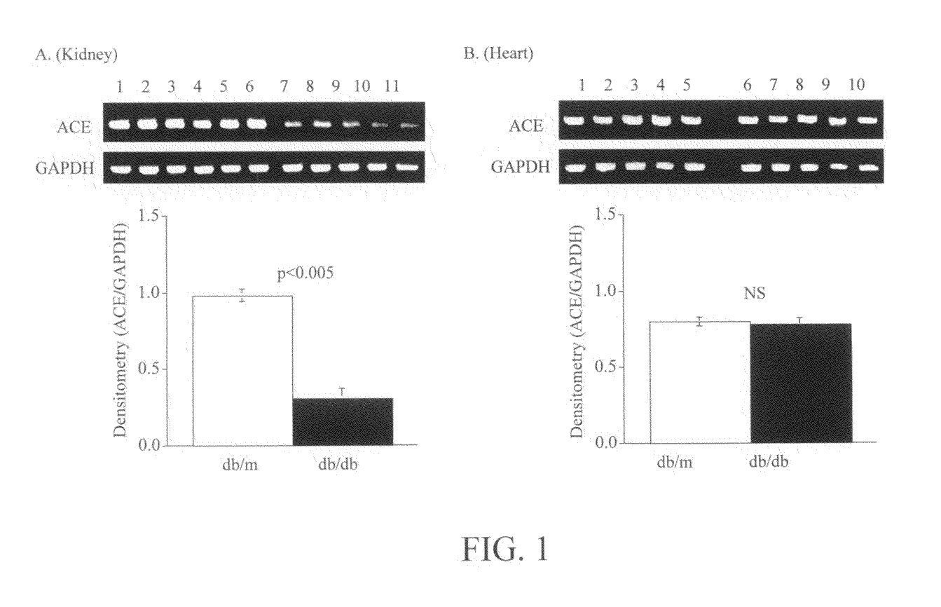

RT-PCR. Tissue levels of ACE mRNA were determined by semi-quantitative RT-PCR after normalization against GAPDH. A single transcript of 339 bp as amplified for ACE and 624 bp for GAPDH (FIG. 1). ACE:GAPDH ratio in renal cortex from db/db mice (n=5) was markedly lower than that observed in db/m controls (n=6) (db/db 0.31±0.06 vs. db/m 0.99±0.05, p<0.005; FIG. 1A). In contrast, ACE:GAPDH mRNA ratio in heart tissue was not different between db/db mice and control db/m mice (db/db 0.78±0.03 n=5 vs. db/m 0.80±0.03 n=5, NS; FIG. 1B). In lung tissue there were also no significant differences between diabetic and control mice (db/db 0.97±0.11 n=5 vs. db/m 0.91±0.05 n=6, NS).

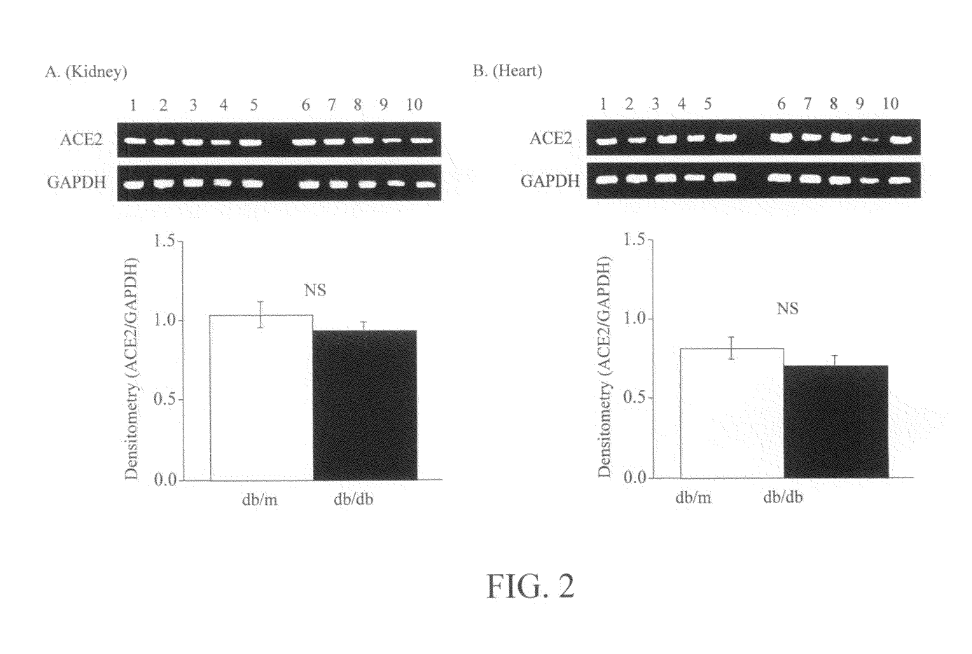

Tissue levels of ACE2 mRNA were only determined in kidney cortex and heart as lung tissue does not appear to express significant amounts of ACE2. A single band at 430 bp was amplified by RT-PCR using ACE2 specific primers (FIG. 2). ACE2:GAPDH ratio in the kidney was not significantly different between diabetic db/db and db/m control mice (db/db mice 0.94±0.05 n=5 vs. db/m 1.03±0.11 n=5, NS; FIG. 2A). Likewise, in the heart, ACE2: GAPDH ratio was similar in db/db and db/m mice (db/db mice 0.70±0.06 vs. db/m 0.81±0.07; NS; FIG. 2B).

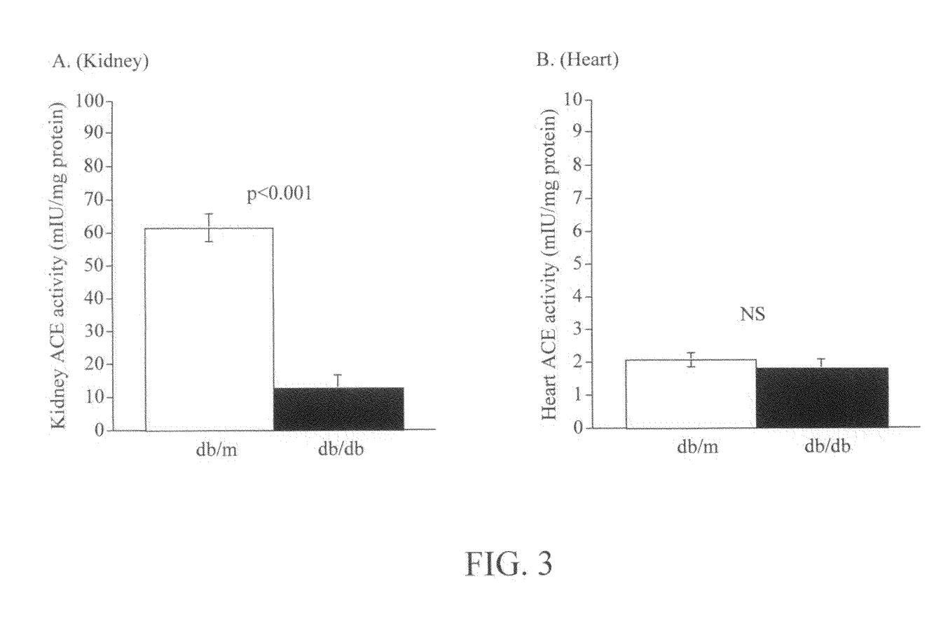

ACE Activity. ACE activity was determined in renal cortex, heart and lung tissue. ACE activity in the renal cortex was markedly decreased in diabetic mice compared to controls (db/db 12.7±3.7 vs. db/m 61.6±4.4 mIU/mg protein, p<0.001; FIG. 3A). In heart tissue, by contrast, ACE activity was similar in db/db and db/m mice (heart: db/db 1.81±0.26 vs. db/m 2.05±0.21 mIU/mg protein, NS FIG. 3B). In lung tissue, ACE activity was the highest but not significantly different between db/db (269.9±32.9 mIU/mg protein) and db/m mice (229.5±19.6 mIU/mg protein). Thus, the reduction in ACE activity in diabetic mice appears to be organ specific for the kidney.

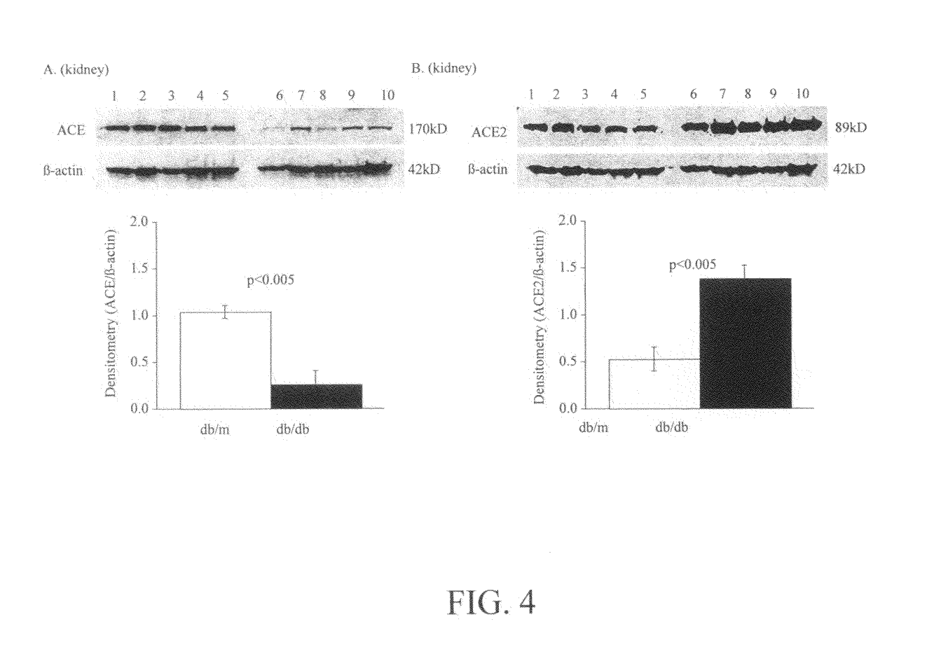

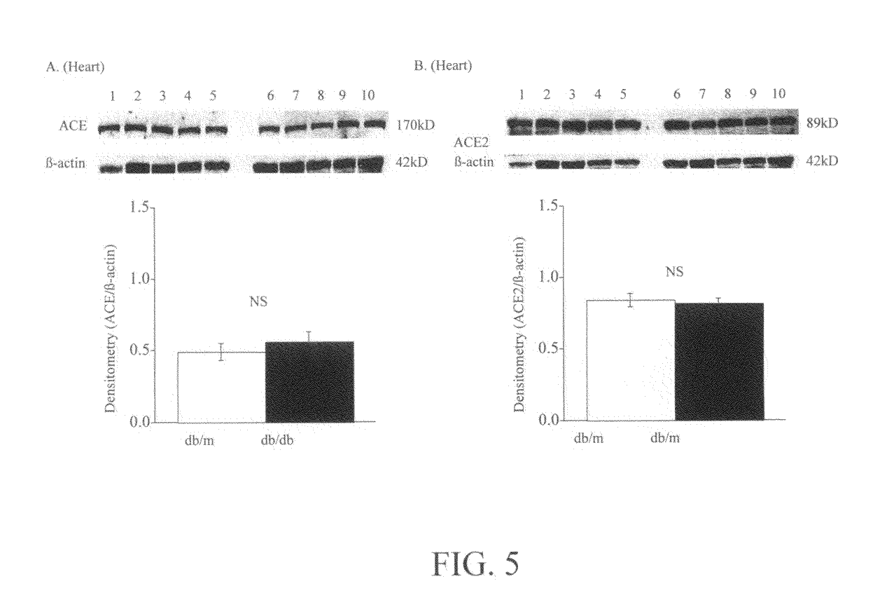

Western Blotting. In kidney cortex and heart tissue, a single band of protein was seen at 170 kDa for ACE and at 89 kDa for ACE2 when membranes were probed with the respective antibodies (FIGS. 4 and 5). These values are consistent with the molecular weights of ACE and ACE2, respectively as reported by others.

ACE protein expression was markedly reduced in kidney cortex of db/db mice as compared to that from db/m controls (db/db 0.24±0.13 n=5 vs. db/m 1.02±0.12 n=5, p<0.005, FIG. 4A). ACE2 protein, by contrast, was higher in kidney cortex of db/db mice than in controls (db/db 1.39±0.14 n=5 vs. db/m 0.53±0.04 n=5, p<0.005, FIG. 4B). In heart tissue, there were no significant differences between db/db and db/m mice in either ACE (db/db 0.56±0.07 n=5 vs. db/m 0.49±0.06 n=5) (FIG. 5A) or ACE2 protein abundance (db/db 0.72±0.07 n=5 vs. db/m 0.79±0.11 n=5) (FIG. 5B).

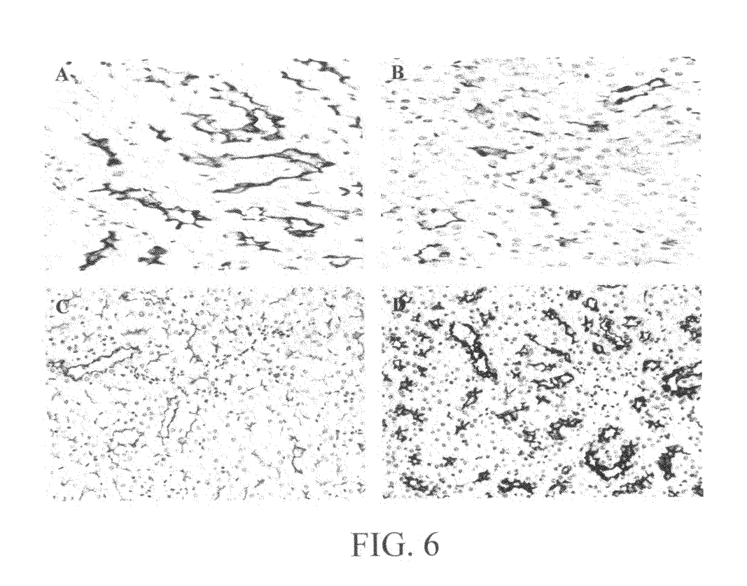

Immunohistochemistry. Prominent ACE and ACE2 staining was observed in the renal cortex but not in the medulla. Strong staining for both ACE and ACE2 was seen along the lumens of renal cortical tubules (FIG. 6). There was a marked reduction in ACE staining in diabetic mice (FIG. 6B) as compared to control mice (FIG. 6A). By contrast, ACE2 staining in cortical tubules of db/db mice was much more intense than in cortical tubules from the db/m controls (FIGS. 6D and 6C, respectively). These findings are in full accordance with the reduction in ACE protein and the increase in ACE2 protein as determined by Western blotting.

### Example 2

### Localization of ACE and ACE2 within the kidney

After anesthetizing by pentobarbital sodium injection, mice were perfused briefly with ice cold PBS to flush out blood, kidneys were removed and fixed in 10% paraformaldehyde, and processed for paraffin embedding according to standard procedures well known in the art. The morphology was evaluated using hematoxylin and eosin-stained sections.

Antibodies. To localize and identify the pattern of distribution of ACE and ACE2, specific markers to different cell types in the nephron were used. To stain the parietal and visceral epithelium (podocytes), anti-podocin antibodies which present in the basal pole of podocytes and strictly follow the external aspect of the glomerular basement membrane were used as well as anti-nephrin antibodies, which localize specifically in the slit diaphragm. Synaptopodin is an actin-associated protein in the podocyte foot process. PECAM-1 (CD31) is expressed over the entire plasma membrane of endothelial cells, and also stains the periphery of the glomerular tuft. Anti-SMA (smooth muscle actin) antibody was used to stain mesangial cells. Markers for tubules are AQP-2 for colocalization within the principal cells of collecting ducts, and a4 (a4 subunit of H-ATPase) for intercalated cells. PECAM-1 and VWF were used to stain the tunica intima and tunica media of the blood vessel wall respectively. ACE and ACE2 antibodies were used concomitantly with each marker. The primary antibodies used in immunofluorescence staining are summarized in Table 2.

TABLE 2
Primary antibodies used for immunofluorescence staining

| Antibody | Host | Dilution | Provider |
|---|---|---|---|
| ACE2 | rabbit | 1:100 | Dr. Batlle |
| Anti-ACE | rat | 1:50 | Dr. S. M. Danilov |
| Anti-Podocin | goat | 1:50 | Santa Cruz |
| Anti-Nephrin | goat | 1:50 | Santa Cruz |
| Anti-Synaptopodin | mouse | 1:50 | Biodesign |
| Anti-PECAM-1 | goat | 1:50 | Santa Cruz |
| Anti-SMC | mouse | 1:50 | Sigma |
| Anti-AQP-2 | goat | 1:100 | Santa Cruz |
| Anti-VWF | goat | 1:100 | Santa Cruz |
| Anti-a4 | rabbit | 1:100 | Dr. Batlle |

For secondary antibodies, Alexa Fluor 488 (donkey anti-rat), Alexa Fluor 555 (donkey anti-rabbit), and Alexa Fluor 647 (donkey anti-goat IgG) or Alexa Fluor 647 (donkey anti-mouse IgG) from Molecular Probes were used.

Triple Immunofluorescence Staining and Confocal Microscopy.

The kidneys were quickly removed after perfusing with cold PBS, and cut longitudinally, fixed with 10% formalin, and embedded in paraffin sections of about 4 μm were cut and mounted on SUPERFROSET PLUS slides (Fisher Scientific). Sections were rehydrated and antigens were retrieved with a pressure cooker. For antigen colocolization, indirect immunofluorescence staining was performed. Sections were washed three times in PBS and permeabilized with 0.5% Triton-X100 for 5 minutes and blocked with 5% normal donkey serum in PBS for about 1 hour at room temperature. The sections were then incubated with primary antibodies including ACE, ACE2 and one of the specific cell type markers for overnight at 4° C. Primary antibodies were diluted in 5% donkey serum in PBST (0.1% TWEEN-20 in PBS). Sections were washed three times in PBST, and incubated with second antibodies diluted 1:200 in PBST with 5% donkey serum for about one hour at room temperature. After washing three times with PBS, sections were mounted with Prolong Gold antifade reagent (Molecular Probes) to delay fluorescence quenching. After covering with cover slips and sealing with nail polisher, sections were visualized with a Zeiss LSM 510 confocal microscope (Carl Zeiss Microscopy, Germany). Negative staining controls for the double or triple labeling procedures were performed by substitution of non-immune serum for the primary antibodies.

Immunohistochemical Staining. To characterize the difference in expression of ACE and ACE2 in control and diabetic mice, kidneys from db/m and db/db mice were cut and fixed in 10% buffered formalin and embedded in paraffin. Sections (about 4 μm) were deparaffinized in xylene and rehydrated through graded alcohols. Antigen retrieval was performed with a pressure cooker at 120° C. in target retrieval solution (DAKO). Endogenous peroxidase activity was blocked with 3% hydrogen peroxide. Slides were incubated with ACE or ACE2 affinity purified rabbit antibody, washed and incubated with secondary antibody conjugated with peroxidase-labeled polymer (DAKO). After incubation with DAB+chromogen, slides were counterstained with hematoxylin. Sections were dehydrated, covered with PERMOUNT (Fisher Scientific) and a cover slip, and then viewed with a Zeiss microscope.

Statistical Analysis. A semi-quantitative evaluation to assess the levels of the ACE and ACE2 expression in glomeruli was performed with immunoperoxidase staining and by counting 100 glomeruli in each mice kidney section, scoring was follows: 1=no detectable staining, 2=weak staining, 3=strong staining. Statistical analysis was performed by Student t test or ANOVA as appropriate. Statistical significance was defined as p<0.05. Data are expressed as mean±SEM.

General Results. Serum glucose was higher in db/db mice than in db/m (406±51 for db/db compared to 178±11 mg/dL db/m, p<0.005). The average body weight in db/db mice was markedly increased as compared to their lean db/m litermates (34.7 g±0.86 for db/db compared to 19.5 g±0.25 g for db/m mice, p<0.005). Kidney weight was increased in db/db mice compared to db/m litermates consistent with the larger size of the animals (0.128±0.005 for db/db compared to 0.113±0.03 g for db/m mice, p<0.005). Albumin/creatinine ratio was increased in db/db mice when compared to db/m (0.29±0.06 for db/db compared to 0.08±0.02 mg albumin/mg creatinine for db/m mice, p<0.005).

In kidney sections stained with hematoxylin and eosin there were no apparent differences between diabetic and control mice, consistent with previously reports in db/db and db/m mice of 8 weeks of age. There were no discernible differences between db/db and db/m mice regarding the number of mesangial cells or the degree of matrix expansion. The size of the glomeruli, however, were increased in db/db mice, a finding also previously noted at an early age in the db/db mice. The glomerular basement membrane in the db/db mice was not thickened and there was no evidence of arteriolar hyalinosis, tubulointerstitial fibrosis, or atrophy.

Immunohistochemical Staining of Control and Diabetic Mice Kidney. The apical border of proximal tubules stained for both ACE and ACE2. In tubules from diabetic mice, proximal tubular staining for ACE was less intense than in tubules from the db/m mice. By contrast, ACE2 staining in tubules from the diabetic mice was increased as compared to control mice.

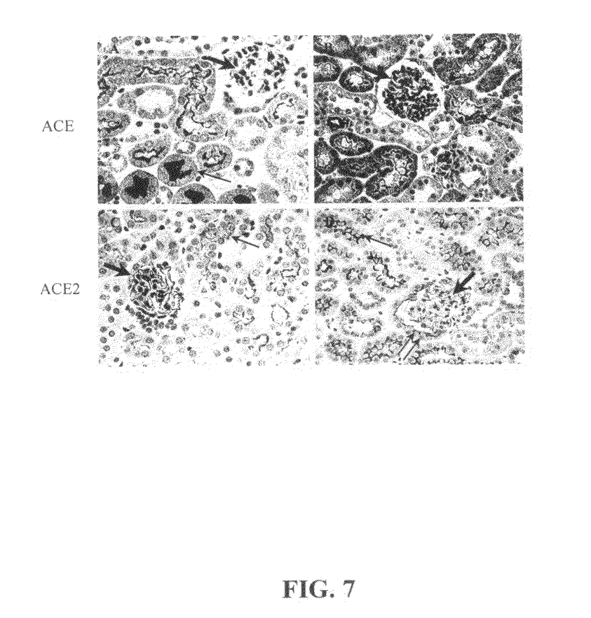

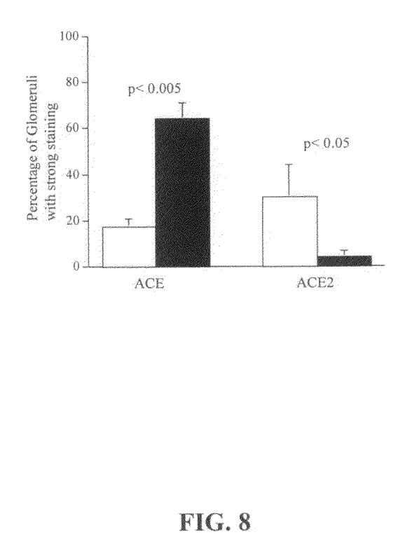

Glomeruli from db/db mice and db/m stained for both ACE and ACE2, but the pattern of staining was just the opposite of what was observed in proximal tubules. ACE staining was increased in glomeruli from db/db mice as compared to db/m (FIG. 7, compare Panel A to Panel B). In an effort to quantify this apparent difference, a visual scale of (1) absent/weak, (2) intermediate and (3) strong was used, and multiple readings were made independently by three blinded observers. Kidneys from 6 animals in each group were examined. In glomeruli from diabetic mice, strong ACE staining was more frequently seen than in glomeruli from control mice (db/db 64.6%±6.3 vs. db/m 17.8%±3.4, p<0.005) (FIG. 8).

In contrast to the above findings with ACE, the percentage of glomeruli expressing strong ACE2 staining was reduced in diabetic mice in comparison to controls (db/db 4.3%±2.4 vs. db/m 30.6%±13.6, p<0.05) (FIG. 8). The percentage of glomeruli with intermediate ACE staining intensity was significantly decreased in kidneys from db/db mice (db/db 34.1±4.2 vs. db/m 69.3±5.3%, p<0.005). Weak staining was the pattern seen less frequently in glomeruli from db/db and db/m (1.2±0.7 and 13.0±3.5%, respectively p<0.005). The percentage of glomeruli showing intermediate or weak ACE2 staining was not significantly different between db/db and db/m mice (50.5%±13.2 vs. 41.1%±12.4 NS and 45.2%±14.4 vs. 28.3%±17.5, respectively).

As in the proximal tubules, parietal epithelial ACE2 staining was increased in glomeruli from the db/db mice (FIG. 7). There was no ACE staining in parietal glomerular epithelium from either db/db or db/m mice.

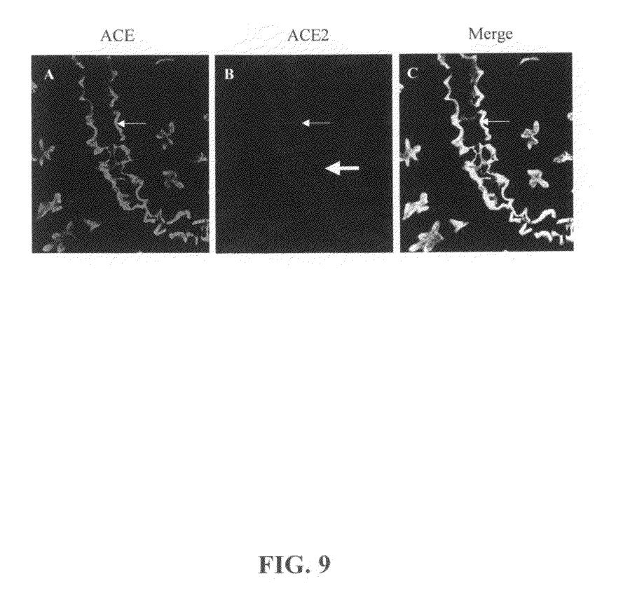

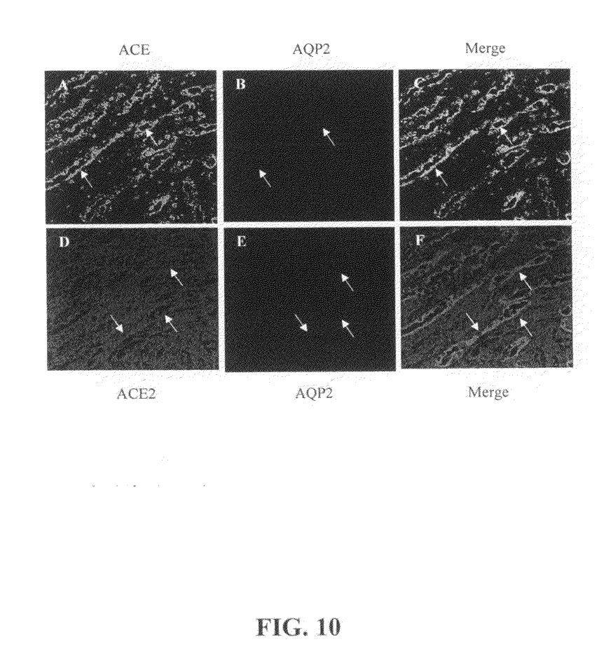

Localization of ACE and ACE2 Using Confocal Microscopy. ACE and ACE2 colocalized strongly in the apical brush border of the proximal tubule. While ACE appears restricted to the apical border, ACE2 was also expressed, albeit weakly, in the cytoplasm. ACE2 is also weakly present in the cytoplasm of proximal and distal tubules (FIG. 9). In collecting tubules, ACE2 colocalized strongly with AQP-2, indicating ACE2 expression in principal cells (FIG. 10). ACE also colocalized with AQP-2, but more weakly than ACE2.

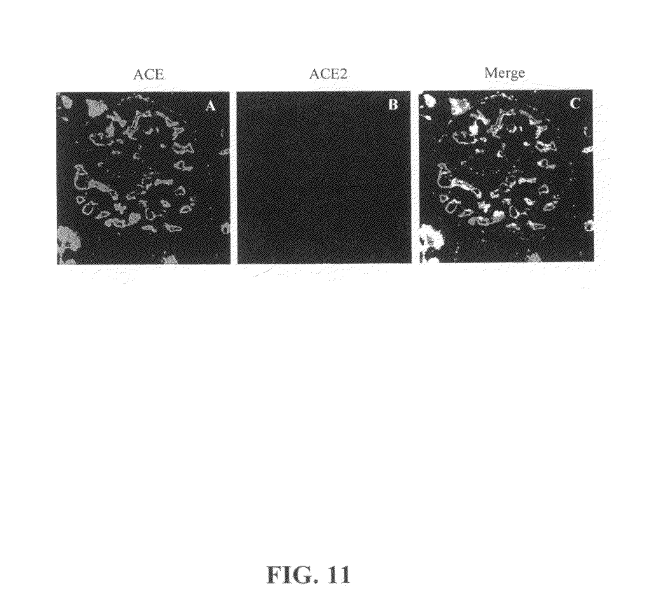

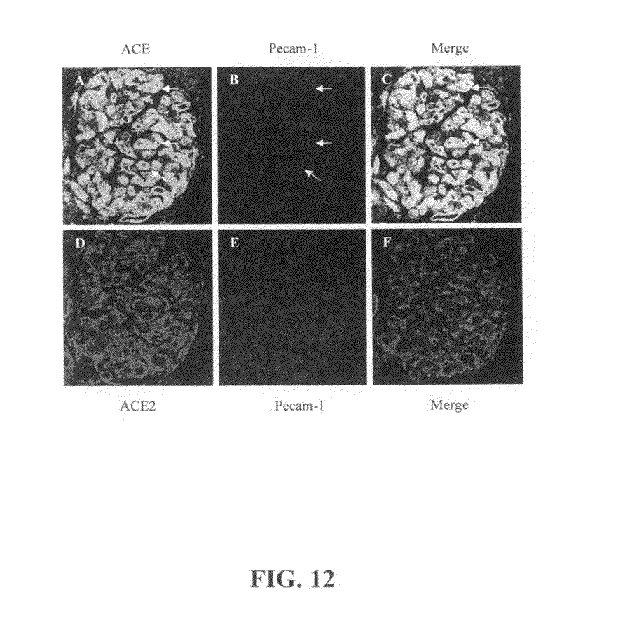

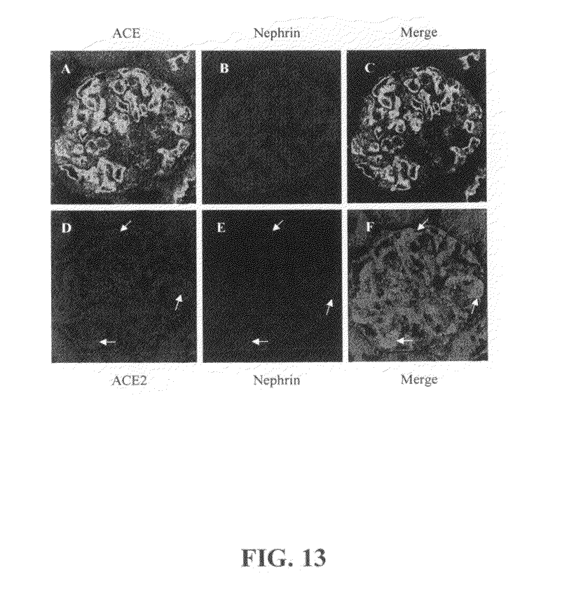

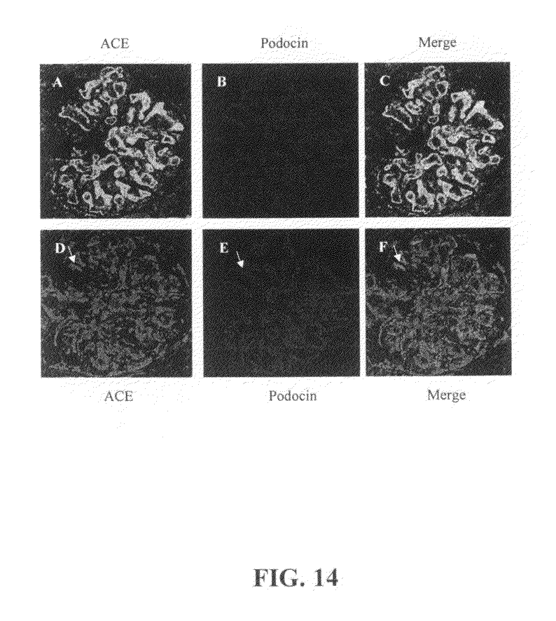

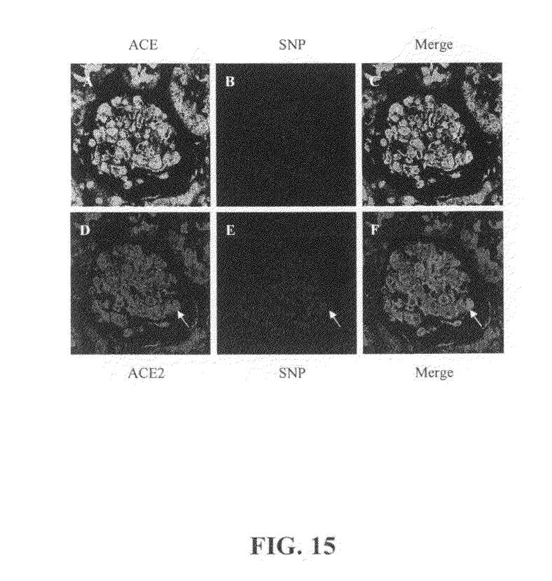

In glomeruli, there was no colocalization between ACE and ACE2 (FIG. 11). To localize each one of those proteins within the glomerular structures, markers for epithelial, mesangial and endothelial cells were used. ACE colocalized with PECAM-1, an endothelial cell marker (FIG. 12, upper panels), whereas ACE2 did not (FIG. 12, lower panels). ACE did not colocalize with nephrin (FIG. 13), podocin (FIG. 14), or synaptopodin (FIG. 15). In contrast, ACE2 colocalized with nephrin, podocin, and synaptopodin. Colocalization of ACE2 with podocin, however, was weak as compared to nephrin and synaptopodin. Neither ACE nor ACE2 colocalized with mesangial cells. In summary, ACE2 is localized in visceral epithelial cells (podocytes) and colocalizes strongly with nephrin, a slit diaphragm protein, and synaptopodin (a foot process protein), ACE2 does not colocalize with an endothelial marker, whereas ACE does.

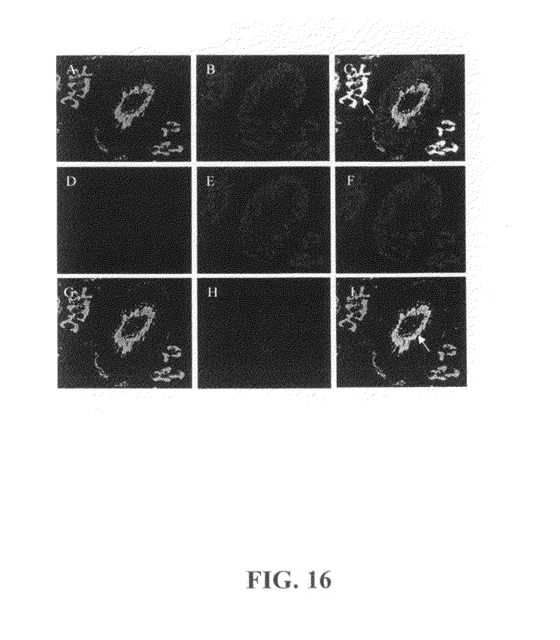

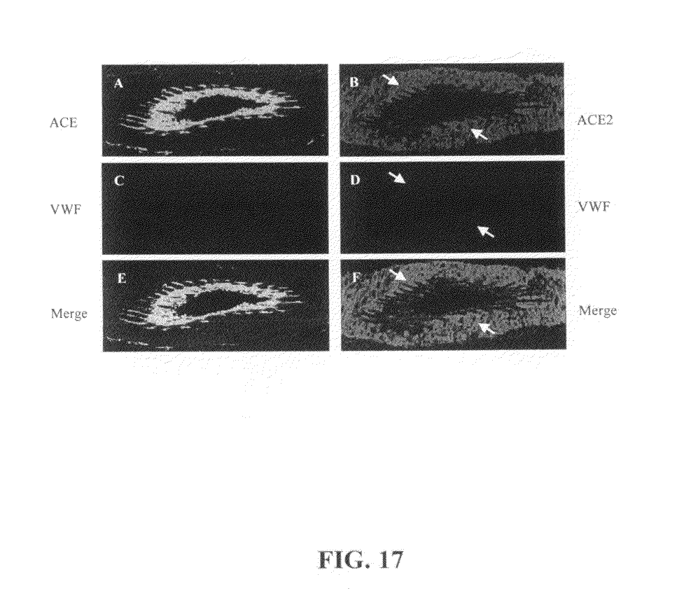

In renal blood vessels, there was no colocalization between ACE and ACE2 (FIG. 16, upper panel). This is in sharp contrast to colocalization seen in proximal tubules (FIG. 7). ACE colocalized with PECAM-1 indicating its presence in the endothelial layer (FIG. 16, lower panel). ACE2, by contrast, did not colocalize with PECAM-1 in renal vessels (FIG. 16, middle panel). ACE2 colocalized with vWF, suggesting a location in the tunica media, whereas ACE did not (FIG. 17).

### Example 3

### Achieving a Protective Level of ACE2 within the Kidney

Pharmacological ACE2 inhibition in db/m and db/db mice. A specific ACE2 inhibitor, MLN-4760 (kind gift from Millennium Pharmaceuticals, Cambridge, Mass.) was injected to db/m and db/db mice subcutaneously (40-80 mg per kg of body weight (mg/kg/BW), every other day), starting at 8 weeks of age until the mice reached the age of 24 weeks. Vehicle control mice received injections of sterile PBS in the same volume. A group of db/db mice received both the AT1 receptor antagonist, telmisartan (Boehringer Ingelheim), in drinking water in a dose of 2 mg/kg body weight/day, as well as the subcutaneous injections of MLN-4760.

Urinary albumin/creatinine ratio. ELISA kits for murine urinary albumin and creatinine companion kits from Exocell were used according to manufacturer's instructions to measure albumin/creatinine ratio in urine samples. Spot urine samples were collected at 8 weeks of age, before initiating the administration of the ACE2 inhibitor, and the AT1 blocker (at 8 weeks of age) and after 12 and 16 weeks of administration of these agents.

Statistical analysis. Statistical analysis was performed using unpaired t-test or ANOVA when appropriate. Significance was defined as p<0.05. Data were expressed as mean±SE.

The Effect of chronic ACE2 inhibition on albumin excretion and glomerular fibronectin deposition. To determine the extent to which chronic ACE2 inhibition results in increased albuminuria in the db/db mice, a specific ACE2 inhibitor, MLN-4760, was administered to the mice for 16 consecutive weeks starting at 8 weeks of age. At 8 weeks of age, before starting MLN or vehicle administration, db/db mice from both groups had virtually indistinguishable levels of urinary albumin excretion (UAE; 69.5±18 vs. 81±15 μg albumin/mg creatinine, respectively). At this age, albumin excretion is already significantly higher in the db/db than in the db/m mice (81±15 vs. 45±5 μg albumin/mg creatinine, respectively). In db/db mice receiving MLN-4760, albumin/creatinine ratio was significantly higher than in their vehicle-treated counterparts after 12 weeks of treatment (474±166 vs. 124±23 μg/mg, respectively, p<0.05). After 16 weeks of treatment, at the age of 24 weeks, MLN-treated db/db mice had about a three-fold increase in UAE in comparison to vehicle db/db controls (743±200 vs. 247±53.9 μg/mg p<0.05, respectively). In db/m mice treated with MLN-4760, UAE was higher than in the vehicle-treated db/m controls, but the difference was small and not statistically significant (55±24 vs. 32±3, μg/mg, respectively, p=NS).

Both MLN-4760 and telmisartan (an ANG II inhibitor) were administered to db/db mice to assess the level to which ANGII inhibitors can ameliorate suppressed ACE2 levels and the proteinuria associated therewith. The administration of telmisartan to diabetic mice completely prevented the increase in urinary albumin associated with administration of the ACE2 inhibitor. This result demonstrates that the effect of ACE2 inhibition on proteinuria requires stimulation of the AT1 receptor, e.g., by increased levels of angiotensin II.

Chronic ACE2 inhibition was also associated with an increased glomerular deposition of fibronectin, an extracellular matrix protein. In glomeruli from db/m mice receiving MLN-4760, fibronectin staining was increased as compared to their vehicle-treated db/m counterparts. In db/db mice, the MLN-4760 administration was also associated with an exaggeration of fibronectin staining. The number of glomeruli with strong fibronectin staining was used to semi-quantify the observed changes in kidneys from 12-16 animals in each group. In db/m mice receiving MLN-4760, the percentage of glomeruli with strong fibronectin staining was increased as compared to glomeruli from vehicle-treated db/m controls (41.1%±4.1 vs. 17.3%±5.2, respectively, p<0.005). Similar to the findings in db/m mice, the percentage of glomeruli with strong fibronectin staining was increased in diabetic mice treated with MLN-4760 in comparison to the db/db mice receiving vehicle (54.8%±4.6 vs. 28.5%±6.4, respectively, p<0.005).

### Example 4

### Concurrent Assay for ACE and ACE2 activity in a Tissue

A fluorescent substrate of ACE and ACE2, i.e., 7-Mca-YVADAPK(Dnp) (R&D Systems), was used to concurrently assay ACE and ACE2 protein activity in tissue samples. Cleavage of this substrate by either enzyme removes the 2,4-dinitrophenyl moiety that quenches the fluorescence of the 7-methoxycoumarin moiety, thus resulting in increased fluorescence. To prevent undesirable hydrolysis of the substrate by a range of non-metalloprotease enzymes from mouse tissues, all tests were performed with the addition of an inhibitor cocktail (complete EDTA-free tablets, Roche).

The ACE inhibitor captopril (ICN) and a carboxypeptidase A inhibitor, benzyl succinate (Sigma), failed to quench fluorescence when incubated with human recombinant (hr) ACE2 (20 nmol/L, R&D systems) at a concentration up to 100 μmol/L. The effects of two different ACE2 inhibitors, MLN-4760 (Millennium Pharmaceuticals Inc.) and DX600 (Phoenix Pharmaceuticals), were examined at concentrations ranging from 100 μmol/L to 100 μmol/L on hrACE2. MLN-4760 quenched the signal completely at 1-10 nmol/L, whereas a higher concentration (100 nmol/L) of DX600 was needed to achieve complete quenching of the signal. Therefore, further studies were done using MLN-4760. In tissue extracts, the concentrations of MLN-4760 required for fluorescence quenching were high and more variable. Near maximal inhibition of the fluorescence signal, calculated per μg of total protein, was achieved at a concentration of MLN-4760 ranging from 10 μmol/L to 1 mmol/L.

Tissue samples (kidney cortex and heart) were homogenized in a buffer consisting of (in mmol/L) 50 HEPES, pH 7.4, 150 NaCl, 0.5% Triton X-100, 0.025 ZnCl2, and 1.0 phenylethanolamine-N-methyltransferase (PMSF), and then clarified by centrifugation at 10,000 g for 15 minutes. After measuring protein concentration, tissue samples were diluted in a buffer [50 mmol/L MES (4-morpholineethanesulfonic acid), 300 mmol/L NaCl, 10 μmol/L ZnCl2, and 0.01% Triton-X-100 pH 6.5], containing EDTA-free tablets. To each well, 88 μl of a diluted tissue sample (1 μg of total protein/well for kidney tissue extracts; 10 μg/well for heart tissue) was added, along with 10 μL of buffer (with the respective inhibitor) and the reaction was initiated by the addition of 2 μL of the substrate (1.0 μmol/L, final concentration). The plates were read using a fluorescence plate reader FLX800 (BIOTEK Instruments Inc.) at an excitation wavelength of 320 nm and an emission wavelength of 400 nm. All reactions were performed at ambient temperature in microtiter plates with a 100 μl total volume.

Kidney cortex samples were incubated at room temperature to assess the time-dependency of the fluorescence signal. As with other fluorophores, the signal resulting from 7-Mca-YVADAPK(Dnp) hydrolysis, increased with time. Fluorescence readings both in the absence and in the presence of MLN-4760 (1 mmol/L) and captopril (10 μmol/L) were near maximal after 4 hours of monitoring and therefore this time-point was chosen for the studies. It is preferred that the assay be run at this time point, because after four hours subsequent digestion of the products of ACE and ACE2 activity occurs and therefore may interfere with the enzyme activity measurements in tissue samples.

Background fluorescence readings over time were obtained from reactions without tissue samples. No substantial increase in fluorescence was noted even after 24 hour incubation, indicating that there is no significant spontaneous substrate hydrolysis under the reaction conditions.

In one set of experiments, kidney tissue samples lacking ACE2 or ACE obtained from the respective knockout mice were spiked with increasing amounts of exogenous human recombinant ACE2 (R&D Systems) or human ACE standard (ACE Kinetic, Bühlmann Laboratories AG, Switzerland) and the resultant increase in fluorescence was recorded.

Defining Tissue ACE and ACE2 Activity.

7-Mca-YVADAPK(Dnp) is cleaved by purified ACE and ACE2 metalloproteases. An effect of another metalloprotease, carboxypeptidase A (Crx A), on this substrate was ruled out by showing that a specific inhibitor, benzyl succinate (BS, 100 μmol/L), did not reduce significantly the fluorescence signal in kidney cortex. Similar results were obtained for heart tissue. This indicates that CrxA does not interfere with measurement of ACE and ACE2 activity using this substrate in kidney and heart tissue. EDTA (1 mmol/L) quenched the fluorescence down to about 16% of control. EDTA chelates the zinc ion required for metalloprotease activity. ACE inhibition, using captopril, decreased the signal to 24.9±1.1% of the control, whereas the specific ACE2 inhibitor, MLN-4760, reduced fluorescence intensity significantly to 46.4±0.7% of the control. The concomitant use of captopril and MLN-4760 nearly completely quenched the fluorescence signal (7.7±0.9% of control).

To account for the effect of ACE on the fluorogenic substrate, while measuring ACE2 activity, the ACE2-dependent signal was measured in the presence of captopril. Conversely, when measuring ACE activity, tissue samples were incubated with MLN-4760.

ACE and ACE2 activity were defined as follows:

ACE activity=A−C, where A=fluorescence in the presence of the ACE2 inhibitor (MLN-4760) and reflects the ACE2 inhibitor-resistant signal and C=fluorescence in the presence of both, Captopril and MLN-4760 and is a reflection of both ACE2 and ACE inhibitor-resistant signals combined).

ACE2 activity=B−C, where B is the fluorescence in the presence of the ACE inhibitor, captopril, and thereby reflects ACE inhibitor-resistant signal; C again is the fluorescence which is resistant to both ACE and ACE2 inhibitor.

The results reported therein are all based on these formulas. The ACE activity can also be defined as the difference between fluorescence without inhibitors and fluorescence remaining after inhibition with the ACE inhibitor, captopril, if desired. Likewise, ACE2 activity can be defined as the difference between fluorescence without inhibitors and fluorescence remaining after inhibition with the ACE2 inhibitor, MLN-4760. There were strong positive correlations for both ACE (r=0.754, n=9) and ACE2 activity (r=0.964, n=9) calculated with each one of the above two formulas for ACE and ACE2. Moreover, in kidney cortex spiked with exogenous ACE and ACE2, both formulas gave similar data in terms of recovery of the respective ACE and ACE2 activity.

Enzymatic activity (RFU/μg protein/hr) was examined in ACE and ACE2 knockout mice and in two rodent models of diabetes, i.e., the db/db and streptozotocin (STZ)-treated mouse models. In kidney cortex, preparations consisting mainly of proximal tubules and cortical collecting tubules, ACE2 activity had a strong positive correlation with ACE2 protein expression (90 kD band) in both knockout models and their respective wild-type littermates (r=0.94, p<0.01). ACE activity, likewise, had a strong positive correlation with renal cortex ACE protein expression (170 kD band) (r=0.838, p<0.005). In renal cortex, ACE2 activity was increased in both models of diabetes (46.7±4.4 vs. 22.0±4.7 in db/db and db/m, respectively, p<0.01; and 22.1±2.8 vs. 13.1±1.5 in STZ-treated vs. untreated mice, respectively, p<0.05). ACE2 mRNA levels in renal cortex from db/db and STZ-treated mice, by contrast, were not significantly different from their respective controls. In cardiac tissue, ACE2 activity was lower than in renal cortex and there were no significant differences between diabetic and control mice (db/db 2.03±0.23 vs. db/m 1.85±0.10; STZ-treated 0.42±0.04 vs. untreated mice 0.52±0.07).

ACE2 activity in renal cortex correlated positively with ACE2 protein in db/db and db/m mice (r=0.666, p<0.005) as well as in STZ-treated and control mice (r=0.621, p<0.05), but not with ACE2 mRNA (r=−0.468 and r=−0.522, respectively). it is clear form the above evaluations that in renal cortex of diabetic mice ACE2 expression is increased at the posttranscriptional level. Enzymatic activity measurements in mouse kidney cortex using two different concentrations of MLN-4760 (10 μmol/L and 1 mmol/L) also showed a good correlation for both ACE (r=0.991, n=6) and ACE2 activity (r=0.858 n=12).

To further examine ACE activity as a function of ACE protein, human ACE standard was added to kidney extract obtained from an ACE knockout mouse. This resulted in an increase in fluorescence signal in a dose-dependent manner with a linear relationship (r=0.988, p<0.001) ranging from 0.0625 to 1.0 mIU/well.

To examine ACE2 activity as a function of ACE2 protein in kidney tissue, purified human recombinant (h)ACE2 protein was added to kidney tissue extract from ACE2-knockout mouse. In these “spiking” experiments the fluorescence signal was recovered in a dose dependent manner, with a linear relationship (r=0.990, p<0.001) in the range of 0.4 to 50 ng ACE2 protein/well. The average activity in tissue extracts from wild type mice corresponded to a concentration of 15 ng hACE2/μg total protein by comparison with purified recombinant enzyme under identical conditions. As little as 0.4 ng of hACE2 was detectable in spiked renal cortex tissue from the ACE2 knockout mouse suggesting an excellent detection limit of ACE2 activity for the present assay method.

To examine the extent to which the increase in ACE2 activity in diabetic mice correlates with hyperglycemia, ACE2 activity was plotted against blood glucose levels. ACE2 activity showed a strong positive correlation with blood glucose levels in STZ mice and their non-diabetic controls pooled together (r=0/863, p<0.0001 for STZ mice). Moreover, a significant positive correlation between ACE2 activity and blood glucose levels was found in kidneys from db/m and db/db mice pooled together (r=0.710, p<0.005 for db/db mice). A positive correlation was also found between blood glucose levels and ACE2 protein in STZ-treated and untreated mice (r=0.647, p<0.01, n=15) as well as db/db and db/m mice (r=0.610, p<0.05, n=15), but not between blood glucose and ACE2 mRNA levels (r=−0.737, p<0.005, n=15; and r=−0.137, NS, n=15, respectively).

ACE/ACE2 activity in heart from diabetic mice was also assessed. Cardiac ACE2 activity was not significantly different between diabetic and control mice (db/db 2.03±0.23 vs. db/m 1.85±0.10; and STZ 0.42±0.04 vs. controls 0.52±0.07 RFU/μg protein/hr). ACE activity measured in the hearts from diabetic mice also did not differ significantly from the respective non-diabetic controls (db/db 2.03±0.37 vs. db/m 2.53±0.30; and STZ 0.630±0.10 vs. non-STZ 0.628±0.07 RFU/μg protein/hr).

The level of ACE2 activity per μg total protein was about 10 to 30 fold higher in kidney cortex than in the heart (see respective values, above). The level of ACE activity was also several fold higher in kidney cortex than in cardiac tissue in both control and diabetic mice. These differences in the level of enzymatic activity between kidney and heart tissue likely reflect that ACE and ACE2 are both abundantly expressed in renal proximal tubules, which represent much of the kidney cortex preparation used in these studies.

The present assay for concurrent measurement of ACE and ACE2 activity provides a useful and needed tool for the evaluation of kidney-specific alterations in the balance of these two carboxypeptidases, which are involved in the control of local angiotensin II formation and degradation.

Intra- and Inter-assay Variability and Comparison with other Methods. The method for measuring enzymatic activity for ACE2 and ACE had intra- and inter-assay coefficients of 14.7% and 10.1%, respectively. The present method was compared to a widely used colorimetric method for measurement of tissue ACE activity (ACE color, Fujirebio). A strong correlation was found between the two methods for ACE activity using renal cortex (r=0.980, p<0.001, n=13).

Discussion

The relative abundance of ACE2 protein determined by Western blotting or by immunostaining was increased in kidney cortex from the db/db mice compared to db/m cortex. ACE protein expression, by contrast, was profoundly decreased in renal tubules from the db/db mice as compared to non-diabetic controls. The reduction of tissue ACE protein expression and the augmentation in ACE2 protein expression in db/db mice were limited to the kidney cortical tubules as no differences were observed between db/db and db/m mice in heart tissue.

The recently identified ACE homolog, ACE2, differs from ACE in that it preferentially removes carboxy-terminal hydrophobic or basic amino acids. ACE2 is highly expressed in kidney and heart. ACE2 appears to be important in cardiac function as its deficiency results in severe impairment of cardiac contractility. To our knowledge, there is no evidence of cardiac dysfunction in the db/db mice in early stages of diabetes. ACE2 mRNA and protein levels in the heart of diabetic mice were similar to control mice, which is consistent with the lack of cardiac involvement at this stage of development of the diabetic condition of db/db mice.

In the db/db mice, the decrease in renal cortex ACE protein expression and increase in ACE2 protein expression detected by Western-blotting were fully concordant with the changes observed by immunostaining of renal cortical tubules. Prominent staining of ACE and ACE2 was observed along the apical surface of cortical tubules in both diabetic and control mice (FIG. 6). The reduction of ACE in renal cortical tubules was unlikely to be caused by the loss of intact renal proximal tubules, which are the site of the highest ACE concentration in the kidney, or ACE-bearing epithelial cells, since kidney histology in diabetic mice did not demonstrate any apparent structural abnormalities. The finding of normal histology is consistent with previous studies in young mice with this model of diabetes. Intrarenal reduction of both ACE and ACE2 reportedly occurs 24 weeks after diabetes induction using STZ. These differences in ACE and ACE2 most likely are due to disease duration and therefore absence of nephropathy at an early age (8 weeks) relative to 24 weeks where nephropathy is already present.

Not wishing to be bound by theory, it is believed that increased ACE2 protein expression in renal cortical tubules from the young db/db mice with early diabetes does not exclude the possibility of an ACE2 reduction later during the course of the disease as nephropathy develops. It is possible that, with time, decreased ACE2 expression combined with increased ACE expression may foster kidney damage in diabetics. ACE2 cleaves ANG Ito form ANG (1-9) and ANG II to form ANG (1-7). ACE2 thus prevents ANG II accumulation, while favoring ANG (1-7) formation. ANG (1-7) has vasodilatory, natriuretic, and antiproliferative actions. Its enhanced formation may have a beneficial effect and counterbalance the deleterious actions of ANG II in terms of kidney damage. Thus, the impact of a low ACE and high ACE2 protein levels on renal angiotensin peptides results in down-regulation of the renal RAS, which is believed to be overactive in the diabetic kidney.

Surprisingly, the finding that in young db/db mice the decrease in ACE activity was associated with an increase in ACE2 protein expression resembles the pattern seen after administration of a renoprotective drug, ramipril, to diabetic rats.

Renal ACE expression in db/db mice was reduced at all levels examined (mRNA, protein and enzymatic activity) and to about the same extent (70-80%), likely reflecting down-regulation at the transcriptional level. Renal ACE2 mRNA, by contrast, was not different from controls, whereas ACE2 protein was clearly increased. The mechanism by which ACE2 protein is increased in the presence of normal mRNA levels was not investigated, although enhanced post-transcriptional processing could explain these observations.

At 8 weeks of age, the diabetic animals in the past study had already developed severe obesity and hyperglycemia. It is unlikely that obesity in the db/db mice is responsible for the finding of suppressed renal ACE expression, because the opposite effect (i.e., a kidney-specific increase in ACE activity) has been reported in obesity prone mice when given a high fat diet.

Low renal ACE activity would be expected to limit ANG II formation, whereas an increase in ACE2 should further prevent ANG II-accumulation by favoring conversion of ANG I to ANG (1-9) and ANG II to ANG (1-7). ANG II overactivity is thought to play a pivotal role in the progression of diabetic nephropathy. The methods of the present invention maintain a renoprotective level of ACE2 expression in the kidneys by administration of an angiotensin II antagonist to a mammal in need of renal protection 2. The resulting decreased renal ACE activity coupled with increased renal ACE2 expression protects the kidneys in the early phases of diabetes by limiting the renal accumulation of ANG II, e.g., by favoring ANG (1-7) formation.

ACE2 is localized in the glomerular podocyte, which is in sharp contrast to ACE, which in the glomerulus is restricted to endothelial cells. In the kidneys of young diabetic db/db mice (8 weeks of age), the pattern of both ACE and ACE2 distribution differ strikingly from that seen in their lean counterpart, the db/m mice. In glomeruli from kidneys of diabetic mice, ACE2 protein expression by immunostaining is attenuated whereas ACE expression is increased. In renal proximal tubules, by contrast, ACE is decreased whereas ACE2 immunostaining is increased.

The location of ACE and ACE2 within glomeruli and other nephron segments were characterized using subcellular and cell-type specific markers by immunofluorescence staining and confocal microscopy. ACE was found to be located within the glomerular endothelial network. ACE2, by contrast, was expressed both in the visceral epithelial cells (podocytes) and in parietal epithelial cells of the Bowman's capsule. Within the podocyte, ACE2 colocalized with nephrin (a slit diaphragm protein) and synaptopodin (a foot process marker) a pattern strongly indicative for ACE2 localization in the podocyte. Based on the observation that ACE2 is not present in either mesangial or endothelial cell, the reduction in glomerular expression of ACE2 observed by immunohistochemistry reflects a decrease in protein content at the level of the podocyte/slit diaphragm complex.

The pattern of excessive ACE and decreased ACE2 expression in db/db mice fosters ANG II accumulation in the glomerulus. The db/db mice at the age of 8 weeks had no evidence of glomerular lesions by light microscopy. In this early age, albumin excretion was already four fold higher in the db/db than the db/m. This increase in albumin excretion reflects an increase in glomerular permeability related to changes in glomerular hemodynamics, subtle podocyte injury, or both.

The location of ACE2 within the podocyte/slit diaphragm complex is protective against ANG II-mediated increases in glomerular permeability. ACE2, by promoting ANG II degradation to ANG 1-7, reduces the amount of ANG II to which the podocyte is exposed. Whether the source of ANG peptides is systemic, from paracrine sources or locally generated within the podocyte, ACE2 provides renoprotection due to its action on ANG II degradation to ANG 1-7 and ANG I degradation to ANG 1-9. Accordingly, ACE2 activity at the level of the podocyte/slit diaphragm complex exerts a renoprotective effect by favoring the rapid degradation of angiotensin peptides, and therefore prevents exposure to high levels of ANG II at the level of the slit diaphragm.

Podocytes in culture produce ANG II by a mechanism that appears to be non-ACE dependent. For instance, in this model, attempts to block ACE with captopril did not abrogate the stretch-induced increase in ANG II generation suggesting a role for non-ACE pathways. The lack of ACE expression in glomerular epithelial cells indicates that the ANG II to which the podocyte is exposed must be either generated by an ACE-independent mechanism or produced outside the podocyte, or both. Regardless of how ANG II is generated within the podocyte, or the source of this peptide (systemic, paracrine), the availability of ANG II within the podocyte/slit diaphragm complex increases glomerular permeability and/or induces glomerular injury. The presence of ACE2 in this critical area of the glomerulus can have an important counter-regulatory role by preventing ANG II accumulation. By the same token, the reduction in glomerular ACE2 observed in diabetic mice can be deleterious by favoring ANG II accumulation. Targeted therapy to amplify ACE2 expression by the methods of the present invention provides a way to prevent proteinuria and confer renoprotection early in the course of diabetic and possibly non-diabetic kidney diseases.

The relative ACE and ACE2 levels in the glomerulus are in contrast with the findings in renal cortical tubules, where ACE staining was decreased but ACE2 was increased. The differences in protein abundance in both ACE and ACE2 between db/db and db/m in renal cortical tubules is demonstrated by Western blot analysis. In the tubules, ACE and ACE2 strongly colococalized on the apical surface on the proximal tubular cells, the main site of ACE and ACE2 expression. However, faint ACE2 staining was also found in the cytoplasm of the proximal and collecting tubule cells. Taken together, there are regions of the nephron with high degree of ACE and ACE2 colocalization (brush border of the proximal tubules) and areas where ACE and ACE2 do not colocalize, but are in a close spatial proximity to each other (glomerulus, vasculature). Accordingly, ACE and ACE2 influence the balance of the angiotensin metabolism in vivo, they do so not only by a direct spatial interaction, but also through a more distant paracrine interaction within different nephron sites or between cell types in a given nephron site.

There is abundant ACE protein in the endothelium of the interlobular arteries in mice. In addition, ACE was observed in the adventitia of renal blood vessels. An augmentation of endothelial ACE has been reported for kidney vessels of diabetic rats. The increase in ACE seen in intimal layer of interlobular arteries is in accordance with an increase of ACE in the endothelial capillaries seen in glomeruli in Example 2. Thus, ACE increases reflect changes within a broader range of renal vessels, from capillaries to arteries. The ACE over-expression seems to be a universal finding in diabetic glomeruli, since an increase in glomerular ACE expression was described previously in rats made diabetic with streptozocin (STZ) and in diabetic patients with nephropathy. As already mentioned, by confocal microscopy the signal for ACE strongly overlapped with that of PECAM-1, the endothelial cell marker. An increase in ACE expression in vessels and in glomerular endothelial cells in diabetic animals and humans can result from generalized endothelial dysfunction, which is increasingly recognized in early stages of diabetes, which can be related to hyperglycemia causing oxidative stress. Hyperfiltration, which is already present at an early age in the db/db mice could play an additional role at the level of the glomerular endothelium. Excessive ACE expression could be the initiating event in the activation of the RAS in diabetes and therefore play a more proximate role than generally suspected. Transgenic mice with either 1, 2 or 3 copies of ACE have been studied after induction of diabetes with streptozocin. After induction of diabetes, there was a moderate but significant increase in urinary albumin excretion (UAE) in 1 and 2 copy mice, but a large increase in UAE in the 3 copy ACE mice.

In summary, the presence of ACE2 in glomerular podocytes plays an important counter-regulatory role by preventing ANG II accumulation. The reduction in glomerular ACE2 observed in diabetic db/db mice can be deleterious by favoring ANG II accumulation which is up to increase glomerular permeability early on and foster progressive injury with duration of hyperglycemia.

The methods of the present invention provide an increase in the cortical tubular ACE2/ACE ratio, resulting in vascular protection, particularly protection of renal vasculature in early diabetes. The opposite pattern (low ACE2 and high ACE) seen in the glomeruli suggests that renal vascular injury is more apt to occur at the glomerular level.

The compound MLN-4760 is a specific ACE2 inhibitor, which exerts its inhibitory action by binding to two metallopeptidase catalytic subdomains of the ACE2 enzyme. To examine the role of ACE2 enzyme in the development of albuminuria we administered MLN-4760 for several weeks. This resulted in a significant increase in albumin excretion in the db/db mice. By 24 weeks of age, albumin excretion was about three-fold higher in db/db mice treated with MLN-4760 as compared to vehicle treated db/db controls. The specific ANGII antagonist, telmisartan, an AT1 blocker, prevented the increase in urinary albumin excretion associated with MLN-4760, indicating that the effect of ACE2 inhibition is mediated by angiotensin II via stimulation of the AT1 receptor. ACE2 inhibition was also associated with increased glomerular expression of fibronectin in both db/m and db/db mice (FIG. 9). In a normal kidney, fibronectin is present along the basement membranes. During glomerular injury fibronectin deposition increases and this increase is considered a marker of extracellular matrix accumulation. Glomerular fibronectin accumulation occurs as early as 7 days after angiotensin II infusion. MLN-4760, by inhibiting ACE2, leads to increased extracellular matrix deposition by promoting ANG II accumulation within the glomerulus. Tt has been reported that in Ace2 knockout mice, ANG II is either endogenously elevated or increased above the levels of wild-type mice after infusion of exogenous ANG II.

The present finding that ACE2 inhibition did not increase albumin excretion significantly in non-diabetic female mice is in keeping with work reported by Oudit et al. using an Ace2 knockout (Am. J. Pathol. 2006; 168:1808-1820). These authors reported that deletion of the Ace2 gene was associated with the development of albuminuria over time (twelve months of age) in male, but not in female mice. In general, it is more difficult to produce albuminuria in female than in male mice. In this respect, it is worthy of note that in the present study female db/db mice were used. The present finding that in female mice ACE2 inhibition resulted in worsening of albuminuria further indicates the importance of this enzyme in the control of the glomerular permeability. Based on this finding, it is likely that in male mice, ACE2 inhibition would promote albuminuria to a greater degree, and that this would affect non-diabetic mice, as well as diabetic mice. It is also of interest to note that ACE2 inhibition in female db/m mice did not result in significant albuminuria despite a significant increase in glomerular fibronectin staining, a marker of mesangial matrix deposition.

Angiotensin II impairs the function of glomerular barrier leading to increased protein excretion. Agents interfering with angiotenisn II activity, such as ACE inhibitors and AT1 blockers, reduce filtration of macromolecules across the glomerular barrier. A recent study reported that the abnormal protein efflux across the glomerular membrane could be mediated by angiotensin II-induced actin cytoskeleton rearrangement in glomerular epithelial cells (Macconi et al., Am. J. Pathol. 2006; 168:1073-1085). The present study demonstrates that the presence of ACE2 in the podocyte/mesangial compartment of the glomerulus can have an important counter-regulatory role by preventing glomerular angiotensin II accumulation. In this respect, the reduction in glomerular ACE2 observed in the young db/db mice could be deleterious, since angiotensin II degradation via ACE2 is apt to be decreased, particularly when coupled with increased angiotensin II formation driven by augmented ACE activity in endothelial cells. It should be noted that the db/db mice at the age of 8 weeks showed no evidence of glomerular lesions by light microscopy, but at this early age albumin excretion was already significantly higher in the db/db than in the db/m mice. This increase in albumin excretion reflects an increase in glomerular permeability related to changes in glomerular hemodynamics, subtle podocyte injury or both. Down-regulation of ACE2 appears to play a role by reducing angiotensin II degradation, whereas the increase in endothelial ACE activity further results in excess ANG II. A cross-talk between podocyte and endothelial cells has been recently proposed to explain the effect of VEGF produced in the podocytes on glomerular endothelial permeability. Although not wishing to be bound by theory, it is possible that the effect of ANGII on augmenting glomerular permeability involves increased VEGF mRNA translation.

It is known that there are angiotensin II receptors (e.g., AT1) in glomerular epithelial cells (podocytes) and that ANG II activates signal transduction pathways in these cells. The glomerular podocyte has a local RAS, and mechanical stress reportedly increases ANG II production in conditionally immortalized podocytes. The present study shows that ACE is not present in the podocyte. Whether the source of ANG peptides is systemic, from paracrine sources, or locally generated within the podocyte, ACE2 is important in determining the levels of angiotensin peptides by promoting ANG II degradation to ANG 1-7 and ANG I degradation to ANG 1-9, respectively. A decreased expression of ACE2 protein and an increase in ACE favors angiotensin II accumulation, which in turn, can lead to increased glomerular permeability.

Glomerular ACE2, and most specifically its presence within the podocyte/slit diaphragm complex normally is protective against ANG II-mediated increases in glomerular permeability. ACE2 activity within the glomerulus exerts a renoprotective effect by favoring the rapid degradation of angiotensin peptides and thereby preventing exposure to high levels of ANG II. This is particularly relevant at the level of the podocyte, a cell which may not be programmed to tolerate angiotensin II which would be in keeping with the lack of ACE expression.

The reduced levels of ACE2 observed in the glomerulus are in sharp contrast to what was observed in renal cortical tubules, where ACE staining is decreased but ACE2 is increased. There have been reports of an increase in ACE in the glomerulus of diabetic patients with nephropathy. An increase in ACE expression in glomerular endothelial cells from diabetic animals and humans may be the result of generalized endothelial dysfunction, which is increasingly recognized in early stages of diabetes. Hyperfiltration which is already present at an early age in db/db mice could play an additional role at the level of the glomerular endothelium. Excessive ACE activation appears to be an important event in the activation of the RAS in diabetes and therefore plays a more proximate role than generally appreciated.

The methods of the present invention stimulate a vascular protection level of ACE2 expression particularly in the kidneys of a mammal in need of such vascular protection (i.e., a diabetic mammal). Administering an angiotensin II antagonist to the mammal maintains the ACE2: ACE podocytes and then results in a state of nephropathy.

Although the present invention has been described in detail in terms of preferred embodiments, no limitation of the scope of the invention is intended. The subject matter in which the applicant seeks an exclusive right is defined in the appended claims.

We claim:

1. An method for concurrently assaying ACE and ACE2 activity in a tissue sample comprising:
(a) contacting a first aliquot of a clarified, diluted tissue homogenate with a fluorescent substrate of both ACE and ACE2 in a physiologically acceptable buffer in the presence of a specific ACE inhibitor for a time sufficient to develop a fluorescence signal proportional to ACE2 activity in the first aliquot;
(b) contacting a second aliquot of a clarified, diluted tissue homogenate with a fluorescent substrate of both ACE and ACE2 in a physiologically acceptable buffer in the presence of a specific ACE2 inhibitor for a time sufficient to develop a fluorescence signal proportional to ACE activity in the first aliquot;
(c) measuring fluorescence in each of the first and second aliquots; and
(d) determining the ACE and ACE2 activity in the tissue sample from the fluorescence measured in the first and second aliquots.

2. The method of claim 1 wherein the activity of ACE and ACE2 in the tissue sample is determined by comparison of the fluorescence measured in the first aliquot with a calibration curve of ACE2 activity versus fluorescence, and comparison of the fluorescence measured in the second aliquot with a calibration curve of ACE activity versus fluorescence.

3. The method of claim 1 wherein the substrate for both ACE and ACE 2 comprises 7-methoxycoumarin-Tyr-Val-Ala-Pro-(2,4-dinitrophenyl)Lys (SEQ ID NO: 7).

4. The method of claim 1 wherein the specific ACE inhibitor comprises captopril.

5. The method of claim 1 wherein the specific ACE2 inhibitor comprises (S,S)-2-{1-carboxy-2-[3-(3,5-dichlorobenzyl)-3H-imidazol4-yl]-ethylamino}-4-methylpentanoic acid (MLN-4760).

6. The method of claim 1 wherein the assay is carried out on a plurality of tissue homogenates from a plurality of tissue samples in a multiwell plate.


SEQUENCE LISTING (7 sequences)

SEQ ID NO: 1 - DNA, 19 bp, Artificial Sequence
  PCR Primer
taactcgagtgccgaggtc

SEQ ID NO: 2 - DNA, 18 bp, Artificial Sequence
  PCR Primer
ccagcaggtggcagtctt

SEQ ID NO: 3 - DNA, 21 bp, Artificial Sequence
  PCR Primer
cttcagcactctcagcagaca

SEQ ID NO: 4 - DNA, 21 bp, Artificial Sequence
  PCR Primer
caacttcctcctcacataggc

SEQ ID NO: 5 - DNA, 23 bp, Artificial Sequence
  PCR Primer
ccagtatgactccactcacggca

SEQ ID NO: 6 - DNA, 24 bp, Artificial Sequence
  PCR Primer
atacttggcaggtttctccaggcg

SEQ ID NO: 7 - protein, 5 aa, Artificial Sequence
  peptide inhibitor
YVAPK